(19) United States
(12) Patent Application Publication
(10) Pub. No.: US 2023/0165918 A1
(43) Pub. Date: Jun. 1, 2023

(54) REDUCTION OF CYTOKINE STORM AND PATHOLOGICAL INFLAMMATION INCLUDING CAUSED BY CORONAVIRUS USING SPHAGNUM AND EXTRACTS THEREOF

(71) Applicant: FIGENE, LLC, Houston, TX (US)

(72) Inventors: Pete O'HEERON, Houston, TX (US); Thomas ICHIM, San Diego, CA (US)

(21) Appl. No.: 17/997,674

(22) PCT Filed: May 4, 2021
(86) PCT No.: PCT/US2021/070504

§ 371 Date: Nov. 1, 2022

Related U.S. Application Data
(60) Provisional application No. 63/020,198, filed on May 5, 2020.

(51) Int. Cl. A61K 36/10; A61K 45/06; A61P 31/14

(52) U.S. Cl. CPC A61K 36/10; A61K 45/06; A61P 31/14

(57) ABSTRACT

Disclosed are treatments, compositions of matter, and protocols for reducing, ameliorating, or reversing excess inflammation and/or cytokine storm through administration of Sphagnum and/or extracts thereof. In particular embodiments, a patient at risk for cytokine storm is administered a Sphagnum extract at a concentration sufficient to induce an immunomodulatory change in said patient, including suppression of macrophage activation, reduction of neutrophil activation and inhibition of DNA extracellular trap release. In some embodiments, Sphagnum, or extracts thereof are administered together with other agents to enhance activity in treatment of Covid-19 infection and associated pathologies. In some embodiments, humic acid and/or analogues thereof are administered for reduction of pathological inflammation and/or stimulation of immunity.

This application claims priority to U.S. Provisional Patent Application Ser. No. 63/020,198, filed May 5, 2020, which is incorporated by reference herein in its entirety.

### TECHNICAL FIELD

Embodiments of the disclosure include at least the fields of cell biology, molecular biology, biology, and medicine.

### BACKGROUND

The COVID-19 pandemic requires a variety of therapeutic agents to fulfill the worldwide needs of a global population and also to handle viruses that are prone to mutation.

The organic component of soil, formed by the decomposition of leaves and other plant material by soil microorganisms is called “humus”. The medical applications of humus or derivatives thereof are described in ancient writings of Romans, Chinese and Indians [1]. Although there was always an almost “supernatural” reverence towards various properties of humic derived elixirs, chemical identification of components from this source did not occur until the 1800s when it was found that the compounds of humus, called humic substances are complex organic substances of soil that are formed during what is called humification [2].

Humification is known to occur as an interaction that comprises of natural chemical and microbial activity that transforms the dead remains of living things into humic substances. Besides photosynthesis, humification is the biggest chemical process occurring on earth and is responsible in part for fossil fuel formation. When various life forms perish, they transform into various substances, some of which are humic acids. Sphagnum is a source of humic acids and other inorganic and organic materials.

While there is an interest in medical applications of humic acids and various other components derived from the soil, the previous studies have not been scientifically validated, or have been generally focused on nutritional content and not therapeutic outcomes.

In various types of viral infections, cytokine storm is one of the main causes of acute respiratory distress syndrome (ARDS) [3-15], as well as other causes of morbidities such as disseminated intravascular coagulation [16, 17]. In some cases cytokine storm is also associated with reduction in lymphocyte numbers [5, 18-24]. It has been shown that various inflammatory cytokines such as interleukin 8 [25-39], TNF-alpha [40], interleukin 1 [41-43], and interleukin 6 correlate with negative prognosis of ARDS. Other cytokines associated with cytokine storm include IFN-gamma, IL-18, TGF-beta, IP-10, MCP-1, and MIG [44].

Therefore, it is desirable in the art to develop effective and non-toxic means of suppressing production of these cytokines, as well as downregulating their pathological activity.

### BRIEF SUMMARY

The present disclosure encompasses methods and compositions for treating excessive production of one or more cytokines in an individual. The excessive production of cytokines may result from an infection, including viral infections such as coronavirus infections. In some embodiments, the methods and compositions encompassed herein are used to treat viral infections, including coronavirus infections (such as SARS-CoV-2), or symptoms associated with such infections. Certain embodiments comprise methods and compositions for preventing lung injury of any kind in an individual. The disclosure encompasses methods and compositions for stimulating the production of one or more growth factors, including keratinocyte growth factor (KGF), in an individual. In some embodiments, the issue of cytokine storm, as well as lung protection from the cytokine storm, is addressed by using humic acid, fluvic acid and/or various components of Sphagnum.

Certain methods encompassed herein comprise the use of Sphagnum, Sphagnum extract, a preparation of Sphagnum, a composition derived from Sphagnum, or a combination thereof. The Sphagnum may be of any species in the Sphagnum genus. Methods for making Sphagnum extracts and/or preparations are known in the art. In some embodiments, Sphagnum, Sphagnum extract, a preparation of Sphagnum, a composition derived from Sphagnum, or a combination thereof are administered to an individual to treat one or more of the diseases, infections, symptoms, or a combination thereof encompassed herein. The Sphagnum extract may be any kind of extract, including an ethanol extract of Sphagnum, a methanol extract of Sphagnum, an aqueous extract of Sphagnum, or a combination thereof. The preparation of Sphagnum may be a tolpa peat preparation. The composition derived from Sphagnum may comprise humic acid and/or fulvic acid. Although compositions may be derived from Sphagnum, they may in other cases be obtained in manners other than extraction, such as purchase of commercially available compounds, including at least humic acid and/or fulvic acid.

In some embodiments, Sphagnum, Sphagnum extract, a preparation of Sphagnum, a composition derived from Sphagnum, or a combination thereof and one or more additional therapies are administered to the individual. In any method herein, the individual may have COVID-19, may be at risk for COVID-19, may be suspected of having COVID-19, may be in need of delaying onset, may be in need of reducing severity of COVID-19, or may be in need of preventing COVID-19. The additional therapies may comprise an antiviral therapy, an immunosuppressive therapy, a chelating agent, an NF-kappa B inhibitor, an antimalarial therapy, a cellular therapy, or combination thereof. The antiviral therapy may comprise hydroxychloroquine and/or chloroquine. The immunosuppressive therapy may comprise rapamycin, cyclophosphamide, prednisone (Deltasone, Orasone), budesonide (Entocort EC), prednisolone (Millipred), tofacitinib (Xeljanz), cyclosporine (Neoral, Sandimmune, SangCya), tacrolimus (Astagraf XL, Envarsus XR, Prograf), mTOR inhibitors, sirolimus (Rapamune), everolimus (Afinitor, Zortress), IMDH inhibitors, azathioprine (Azasan, Imuran), leflunomide (Arava), mycophenolate (CellCept, Myfortic), abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), monoclonal antibodies, basiliximab (Simulect), daclizumab (Zinbryta), or a combination thereof. The chelating agent may comprise deferoxamine mesylate.

In some embodiments, the NF-kappa B inhibitor comprises one or more antisense oligonucleotides, decoy oligonucleotides, short-hairpin RNAs, and/or RNA interference compositions targeting at least one gene in the NF-kappa B pathway. In some embodiments, the NF-kappa B inhibitor is a composition selected from the group consisting of Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin2A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic), and a combination thereof.

In some embodiments, a cellular therapy is provided to the individual. In specific embodiments, the cellular therapy comprises mesenchymal stem cells, hematopoietic stem cells, natural killer cells, and/or fibroblasts. The cellular therapy may be autologous, allogenic, or xenogenic with respect to the individual. In some embodiments, the mesenchymal stem cells and/or the fibroblasts are plastic adherent. The mesenchymal stem cells may express one or more particular markers, such as CD73, CD90, and/or CD105. In some embodiments, the mesenchymal stem cells do not express one or more particular markers, such as CD14, CD34, and/or HLA II. The hematopoietic stem cells may express one or more particular markers, such as CD34 and/or CD133. In some embodiments, the hematopoietic stem cells do not express one or more particular markers, such as CD38. The hematopoietic stem cells may differentiate or may be capable of differentiating into myeloid, erythroid, and/or megakaryocytic lineages. The fibroblasts may be derived from any tissue, including skin, fat, bone marrow, cord blood, Wharton's jelly, hair follicle, or a combination thereof.

In some embodiments, an individual is administered Sphagnum, Sphagnum extract, a preparation of Sphagnum, a composition derived from Sphagnum, or a combination thereof and one or more additional therapies comprising a composition selected from the group consisting of remdesivir, pyridostigmine, desferal, hyperimmune plasma, toclizumab, sialidase DAS181, Dapagliflozin, recombinant ACE2, naproxen, Lopinavir/ritonavir, Baricitinib (janus kinase inhibitor), Sarilumab (anti-IL-6 receptor), Ruxolitinib, Acalabrutinib, interferon, Ciclesonide, Anakinra, Umifenovir, Sargramostim, Sildenafil citrate, Tranexamic acid, and Ivermectine, and a combination thereof.

In the affected individual, excessive production of one or more cytokines may be mediated by unrestrained activation of cells selected from the group consisting of monocytes, peripheral blood mononuclear cells, dendritic cells, gamma delta T cells, natural killer cells, and a combination thereof. The cytokines that are excessively produced may be any cytokine, including at least MCP-1, interleukin 1 beta, interleukin 6, d) interleukin 8, interleukin 11, interleukin-18, interleukin-21, interleukin 27, interleukin 33, HMGB-1, and/or TNF-alpha. The excessive production of cytokines may comprise the production of cytokines at a level higher than the normal physiological level of production. In some embodiments, anti-inflammatory cytokines do not significantly control the excessive production of cytokines. The excessive production of cytokines in an individual may comprise, or be diagnosed as, cytokine storm or cytokine release syndrome.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1 shows that humic acid (HA) increases the production of keratinocyte growth factor (KGF) in monocyte, fibroblast, and mesenchymal cell cultures. From left to right, the bars represent control, HA 5 μg/mL; HA 10 μg/mL; and HA 20 μg/mL.

FIG. 2 shows that humic acid (HA) reduces TNF-alpha production in monocyte, peripheral blood mononuclear cell (PBMC), and dendritic cell cultured with LPS. From left to right, the bars represent control, LPS, HA 10 μg/mL, and hydroxychloroquine at 10 μg/mL.

### DETAILED DESCRIPTION

### I. Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The term “administered” or “administering”, as used herein, refers to any method of providing a composition to an individual such that the composition has its intended effect on the individual. For example, one method of administering is by a direct mechanism such as, local tissue administration, oral ingestion, transdermal patch, topical, inhalation, suppository etc.

The term “individual” or “subject” may be used interchangeable, as used herein, and refer to a human or animal that may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, “Sphagnum” refers to any moss of the genus Sphagnum. The Sphagnum may be from any source.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

### II. Particular Embodiments

It is known that humic acid is a component of humic substances, which are the major organic constituents of soil (humus), peat, and coal; humus can also be found in some upland streams, dystrophic lakes, and ocean water. Humic acid is produced by biodegradation of dead organic matter. Humic acid can be a complex mixture of many different acids containing carboxyl and phenolate groups so that the mixture behaves functionally as a dibasic acid or, occasionally, as a tribasic acid. Humic acids can form complexes with ions that are commonly found in the environment creating humic colloids. Fulvic acids are humic acids of lower molecular weight and higher oxygen content than other humic acids. Humic substances in soils and sediments can be divided into three main fractions: humic acids, fulvic acids, and humin. The humic and fulvic acids can be extracted as a colloidal sol from soil and other solid phase sources using basic aqueous solutions of sodium hydroxide or potassium hydroxide. Humin is insoluble in dilute alkali and is therefore not collected by basic extraction. Humic acids may be precipitated from basic solutions by adjusting the pH to 1 with hydrochloric acid, leaving the fulvic acids in solution. This solubility difference at acidic pH is the operational distinction between humic and fulvic acids. Humic and fulvic acids are commonly used as a soil supplement in agriculture, and less commonly as a human nutritional supplement. As a nutrition supplement, fulvic acid can be found in a liquid form as a component of mineral colloids. Fulvic acids are poly-electrolytes and are unique colloids that diffuse easily through membranes whereas all other colloids do not.

Humic and fulvic acids can react with the chemicals used in the chlorination process, which is sometimes used for generating drinking water, to form disinfection byproducts such as dihaloacetonitriles, which are toxic to humans. Any humic acids and/or fumic acids used in embodiments of the present disclosure may be synthesized or may be extracted and/or prepared from any Sphagnum encompassed herein. Methods for synthesizing, extracting, and/or preparing humic acids and/or fumic acids are known in the art. Although water is generally not used for preparing humic and fulvic acids or the supplements described herein, if water is used to prepare or formulate humic and fulvic acids, a pure source of water (without chlorine or chlorination byproducts) may be used [45].

The compositions encompassed herein may be administered to an individual in any suitable way known in the art. In some embodiments, the compositions are administered intravenously, intra-articularly, intraperitoneally, topically, intrarectally, intra-arterially, intramuscularly, subcutaneously or by aerosol inhalant. In some embodiments, compositions encompassed herein are provided to an individual as a food supplement. In some embodiments, a food supplement comprising Sphagnum, Sphagnum extract, humic acid, and/or fluvic acid are provided to an individual.

In some embodiments, humic, fluvic acids, and/or Sphagnum extracts are administered together with one or more various agents that suppress ARDS or a coronavirus infection, including for example, intravenous immunoglobulin [46-48], antibody to interleukin-8 [49], PGE-2 [50], antisense oligonucleotides targeting NF-kappa B [51], intrapulmonary transfection of hsp70 [52], platelet-activating factor acetylhydrolase [53], neutrophil elastase inhibitors [54, 55], recombinant surfactant C [56], glutamine [57-59], histamine inhibition [60], hesperidin [61], beta glucan [62], activated protein C [63-65], licorice flavonoids [66], cell-penetrating peptide nuclear import inhibitor of nuclear factor (NF)-kappaB [67], hemoperfusion with a beta2-microglobulin-selective adsorbent column [68] polymyxin B-immobilized fiber hemoperfusion [69], Growth hormone releasing peptide-2 [70], erythropoietin [71], penehyclidine hydrochloride [72], KGF-2 (FGF-10) [73], antibody to tissue factor [74], paeonol [75], tumor necrosis factor-alpha-derived TIP peptide [76], Osthole, a natural coumarin derivative extracted from traditional Chinese medicines [77], low dose interferon alpha [78], curcumin [79], apolipoprotein A-I mimetic peptide [80], Ethyl gallate [81], aurothioglucose [82], 5′-N-ethylcarboxamidoadenosine adenosine receptor agonist [83], Schisantherin A [84], Punicalagin [85], Emodin [86, 87], quercetin [88], sivelestat [45], imatinib [89], melatonin [90], SDF-1 [91], Astilbin [92], Tr1 cells [93], STAT3 inhibition [94], vitamin C [95], antibodies to IP-10 [96], Lugrandoside [97], Hydroxysafflor Yellow A [98], Parecoxib [99], Diosmetin [100], antibody to ICAM-1 [101], TNF-alpha blocking agents [102], Geldanamycin [103], rhubarb and rhubarb extracts [104], Zinc Finger Protein A20 [105], resolvin D1 [106], GSK-3 inhibition [107], and vaspin [108].

In particular embodiments, an immunosuppressive therapy is administered to an individual. Any immunosuppressive therapy may be used including, for example, cyclosporin (e.g., Cyclosporine A, Sandimmune®, Neoral®, (Novartis), Rapimmune® (American Home Products), FK501 (Fujisawa), CELLCEPT® (Roche, Syntex), IMUREK®, SPANIDIN® and PROGRAF®.

In some embodiments, humic acids, fluvic acids, and/or Sphagnum extracts are administered together with T regulatory cells, or other types of T cells in order to reduce the cytokine storm produced by innate immune system over-activation [109].

In particular embodiments, humic acids, fluvic acids, and/or Sphagnum extracts are administered together with extracorporeal removal of inflammatory cytokines. For example, it has been previously described in the art that dialysis-type approaches can be utilized to remove various soluble mediators from the blood which are associated with inflammation. In some embodiments removal of cytokines such as IL-1, IL-6 IL-8 and/or TNF-alpha is performed prior to, concurrently with, or subsequent to administration of humic acids, fluvic acids, and/or Sphagnum extracts [110].

In particular embodiments, humic acids, fluvic acids, and/or Sphagnum extracts are administered together with attenuated bacteria that can reduce cytokine storm. For example, in a mouse model of severe influenza virus-induced pneumonitis, it was observed that prior nasal administration of an attenuated strain of Bordetella pertussis (BPZE1) provided effective and sustained protection against lethal challenge with two different influenza A virus subtypes. In contrast to most cross-protective effects reported so far, the protective window offered upon nasal treatment with BPZE1 lasted up to at least 12 weeks, suggesting a unique mechanism(s) involved in the protection. Since no significant differences in viral loads were observed between BPZE1-treated and control mice, indicating that the cross-protective mechanism(s) does not directly target the viral particles and/or infected cells. This was further confirmed by the absence of cross-reactive antibodies and T cells in serum transfer and in vitro restimulation experiments, respectively. Instead, compared to infected control mice, BPZE1-treated animals displayed markedly reduced lung inflammation and tissue damage, decreased neutrophil infiltration, and strong suppression of the production of major proinflammatory mediators in their bronchoalveolar fluids (BALFs). These findings thus indicate that protection against influenza virus-induced severe pneumonitis can be achieved through attenuation of exaggerated cytokine-mediated inflammation [111]. The utilization of this bacterial approach combined with Sphagnum and/or extracts thereof are encompassed herein.

In other embodiments the use of sphingosine-1-phosphate receptor agonist, AAL-R is utilized together with Sphagnum and/or Sphagnum derivatives to reduce cytokine storm and/or accelerate resolution of pathology [112].

In one embodiment of the invention, Sphagnum, Sphagnum extract, humic acid, and/or fulvic acid are administered together with protein SERP-1 (a serine protease inhibitor produced by malignant rabbit fibroma virus (MRV) and myxoma virus (MYX) and the subject of U.S. Pat. No. 5,686,409) its analogs and/or biologically active fragments, and optionally in combination with an immunosuppressant useful for treating the clinical manifestations of viral infections (including SARS-CoV-2, also called COVID-19) or associated pathologies caused by cytokine storm.

In specific embodiments, a therapeutically effective amount of Sphagnum, Sphagnum extract, humic acid, or fulvic acid, and SERP-1, SERP-1 analogs or biologically active fragments thereof, and optionally one or more immunosuppressants are co-administered to a subject in need of such treatment for a time and under conditions sufficient to treat a disease, including for example, COVID-19 or symptoms or related conditions thereof, such as pneumonia or ARDS.

The use of SERP-1 has previously been described in the references cited herein, which are incorporated by reference. The present disclosure encompasses the use of SERP-1 by combining such use with Sphagnum, Sphagnum extract, humic acid, or fulvic acid. Without being bound to theory, relevant activities of SERP-1 to the current disclosure include: protease inhibition [113], suppression of responsiveness to inflammatory cytokines [114, 115], inhibition of cytokine synthesis [116-118], suppression of inflammation [119-125], and reduction of endothelial cell activation [126-130]. The pharmacokinetics and metabolism of SERP-1 are known and the following publication is incorporated by reference to assist one of skill in the art in the practice of the invention [131-133].

Acute respiratory distress syndrome (ARDS) is treatable with the compositions and methods of the present disclosure. ARDS is an inflammatory condition characterized by increased capillary permeability, interstitial and intra-alveolar edema, fibrin exudation, and formation of hyaline membrane. Inflammatory cells and mediators including leukocytes, cytokines, oxygen radicals, complement, and arachidonate metabolites damage capillary endothelium and allow fluid and protein to leak across capillaries. The present disclosure encompasses methods and compositions for treating or preventing ARDS of any cause, whether or not it is the result of coronavirus infection of any kind, including of SARS-CoV-2 infection.

The present disclosure is also directed to treatment of systemic shock and many resultant clinical conditions associated therewith in an individual. Systemic shock often occurs as a complication of severe blood loss, severe localized bacterial infection, ischemia/reperfusion trauma and is a major cause of death in intensive care units. Most cases of septic shock are induced by endotoxins (i.e., bacterial cell wall lipopolysaccharides or LPS) from gram negative bacilli or toxins (i.e., toxic shock toxin 1) from gram positive cocci bacteria. The release of LPS in the bloodstream causes release of inflammatory mediators (inflammatory cytokines, platelet activating factor, complement, leukotrienes, oxygen metabolites, and the like) which cause myocardial dysfunction, vasodilation, hypotension, endothelial injury, leukocyte adhesion and aggregation, disseminated intravascular coagulation, ARDS, liver, kidney and central nervous system (CNS) failure. Shock due to blood loss also involves inflammatory mediator release. In each case, inflammatory responses are induced at the original site of trauma, and also in the vasculature and remote vascularized sites.

COVID-19 may be treated or prevented with methods and compositions of the disclosure, including various physiological maladies associated with the invention. For example, COVID-19 is known to induce numerous cardiac alterations. Myocardial ischemia is associated with activation of the complement system that further promotes cardiac injury with the enhancement of a series of inflammatory events. Life-threatening local and remote tissue damage occurs during surgery, trauma and stroke when major vascular beds are deprived for a time of oxygenation (ischemia), then restored with normal circulation (reperfusion). Reperfusion injury is characterized by vascular permeability leading to edema and infiltration of inflammatory cells. Neutrophils contribute significantly to reperfusion damage by generating oxidants or releasing proteases that damage the microvasculature or adjacent tissue. Cell death and tissue damage due to complement and inflammatory cell mechanisms lead to organ failure or decreased organ function. The activation of mediators by a local injury can also cause a remote injury to highly vascularized organs. The compositions and methodologies of the present invention are useful in the treatment of ischemia and reperfusion injury.

In accordance with the present disclosure, the aforementioned disease and injury conditions are treated by administering a therapeutically effective amount of Sphagnum, Sphagnum extract, humic acid, fulvic acid, SERP-1, and/or SERP-1 analog or biologically active fragment thereof optionally in combination with one or more immunosuppressants in a manner consistent with conventional methodologies associated with treatment of the relevant injury or disease condition such as for example, intravenously, intra-articularly, intraperitoneally, topically, intrarectally, intra-arterially, intramuscularly, orally, subcutaneously or by aerosol inhalant in order to treat inflammatory and immune reactions associated with such disease and injury conditions.

The pharmaceutical forms of Sphagnum, Sphagnum extract, humic acids, fulvic acids, SERP-1, or other compositions encompassed herein suitable for delivery (such as by infusion) include sterile aqueous solutions or dispersions and sterile powders for the extemporaneous preparation of sterile injectable solutions or dispersion. In all cases, the form must be sterile and must be fluid to the extent that easy syringability exists. Typical carriers include a solvent or dispersion medium containing; example, water buffered aqueous solutions (i.e., biocompatible buffers), ethanol, polyols such as glycerol, propylene glycol, polyethylene glycol, suitable mixtures thereof, surfactants, or vegetable oils. Sterilization can be accomplished by any art-recognized technique, including but not limited to filtration or addition of antibacterial or antifungal agents, for example, paraben, chlorobutanol, phenol, sorbic acid or thimerosal. Further, isotonic agents such as sugars or sodium chloride may be incorporated in the subject compositions.

### III. Sphagnum

Sphagnum, commonly known as peat moss, is a genus of moss encompassing approximately 380 species. Embodiments encompassed herein may use one or more of the known species of Sphagnum. Sphagnum typically grows in watery habitats. It has a distinct structure comprising the capitulum, main body, and bottom. Sphagnum plants have a main stem from which leafy branches are emitted. Sphagnum species can often be field-identified into four major sub-categories: (1) Acutifolia, (2) Cuspidata, (3) Sphagnum, and (4) Subsecunda. Sphagnum may be most readily found in peat bogs, conifer forests, moist tundra areas, and moorlands.

As described in the embodiments encompassed herein, Sphagnum may refer to any species of the Sphagnum genus, including for example, at least Sphagnum fimbriatum, Sphagnum magellanicum, Sphagnum palustre, Sphagnum rubellum, Sphagnum fuscum, Sphagnum angustifolium, and Sphagnum subnitens.

Sphagnum may be cultured, grown agriculturally, or harvested from natural sources. Sphagnum may be harvested from natural sources, such as a peat bog, and subsequently cultured in vitro. The Sphagnum may be clonally isolated and cultured, using any suitable method known in the art. The Sphagnum may undergo spore sterilization prior to culturing. Sphagnum may be cultured in liquid medium and/or in a bioreactor.

In some embodiments, Sphagnum is processed to extract compositions useful for methods encompassed herein. Sphagnum may be processed by alkaline extraction to isolate humic acids and/or fluvic acids. In some embodiments, one or more extraction solutions comprising sodium hydroxide, potassium hydroxide, sodium carbonate, or a combination thereof is used to extract compositions from Sphagnum. When the sodium hydroxide, potassium hydroxide, and/or sodium carbonate are in separate solutions, the solutions may be used individually or sequentially in any order to extract compositions from Sphagnum. The sodium hydroxide solution, potassium hydroxide solution, and/or sodium carbonate solution may be approximately 0.1 N, 0.2 N, 0.3 N, 0.4 N, 0.5 N, 1.0 N, 1.5 N, 2.0 N, 2.5 N, 3.0 N, 3.5 N, 4.0 N, 4.5 N, or 5.0 N.

In some embodiments, Sphagnum is optionally cleaned and dried, then subject to an extraction liquid. The extraction liquid may be ethanol, methanol, or an aqueous solution. In some embodiments, the Sphagnum is boiled in an extraction liquid. The extraction procedure may be done once, or may be done repeatedly.

Extracts and/or isolations from Sphagnum may be further purified. The extracts and/or isolations may be purified, for example, by filtration, chromatography, crystallization, or distillation.

### IV. Kits

Any of the compositions described herein may be comprised in a kit. In a non-limiting example, the kit comprises Sphagnum (including extracts, compositions, and/or preparations of Sphagnum), one or more cellular therapies, and/or one or more immunosuppressive compositions. The cellular therapies may be of any kind, including fibroblasts, mesenchymal stem cells, or mixtures thereof.

The kits may comprise a suitably aliquoted compositions of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also may generally contain a second, third or other additional container into which the additional components may be separately placed. However, various combinations of components may be comprised in a vial. The kits of the present disclosure also will typically include a means for containing the compositions and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly considered. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the disclosure may also comprise, and/or be packaged with, an instrument for assisting with the injection/administration and/or placement of the ultimate composition within the body of an animal. Such an instrument may be a syringe, pipette, forceps, and/or any such medically approved delivery vehicle.

### V. Examples

### Example 1

### Stimulation of KGF by Humic Acid from Various Regenerative Cell Types

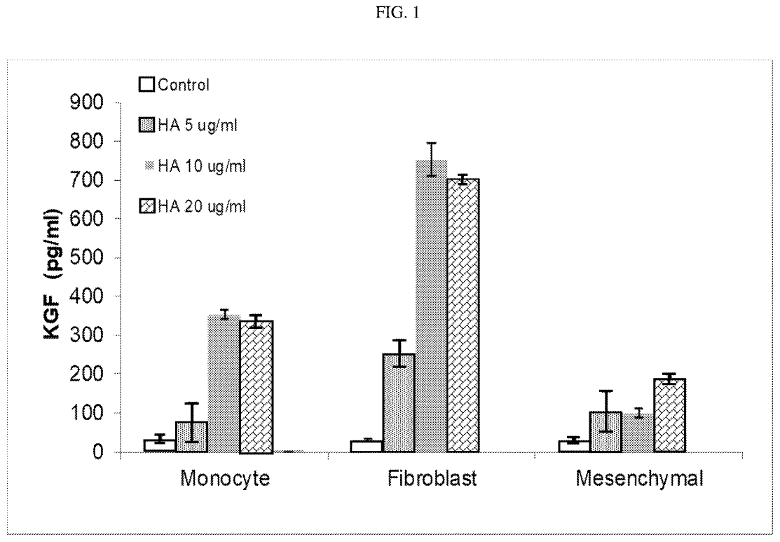

Humic acid (HA) preparation Activomin® (Pharmawerk Weinboehla, Weinboehla, Germany) was administered to adherent cultures of the cells indicated for 48 hours. As seen in FIG. 1, production of keratinocyte growth factor (KGF), as assessed by ELISA, was increased after culture with HA. Therefore, the stimulation of the production of KGF indicates that humic acid may be administered in vivo in therapeutically effective amounts to an individual that has COVID-19 or is at risk for COVID-19 or is suspected of having COVID-19, or for preventing COVID-19.

### Example 2

### Superior Inhibition of TNF-Alpha by Humic Acid Compared to Hydroxychloroquine from Immunologically Relevant Cell Types

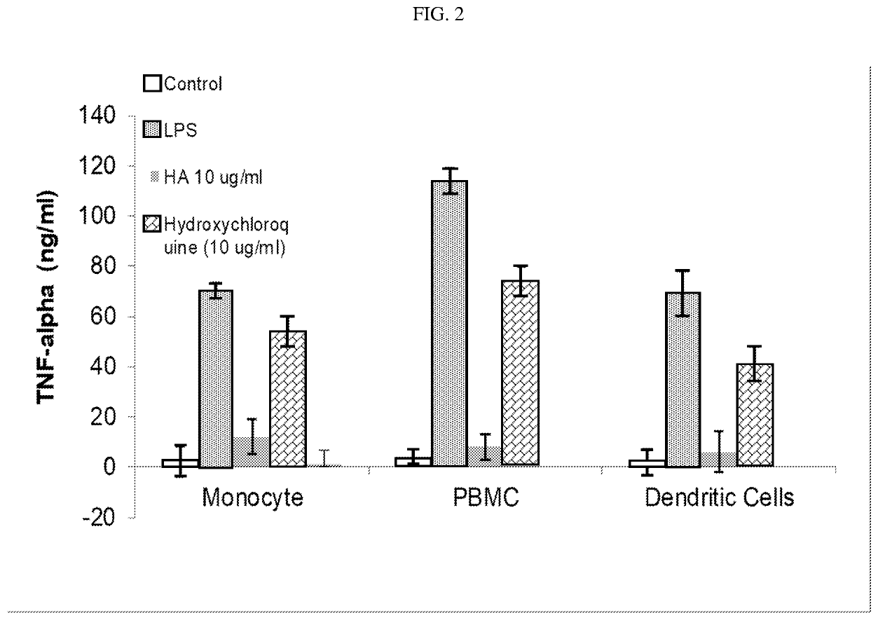

Humic acid (HA) preparation Activomin® (Pharmawerk Weinboehla, Weinboehla, Germany) was administered to adherent cultures of the indicated cells for 48 hours, whereas other of the cells were stimulated with LPS 5 μg/mL. As seen in FIG. 2, production of inflammatory cytokine TNF-alpha, as assessed by ELISA, was decreased after culture with HA. Interestingly, HA was superior to hydroxychloroquine under these conditions. Therefore, HA was able to reduce the cytokine TNF-alpha, and in specific embodiments such reduction occurs in vivo upon delivery of a therapeutically effective amount to an individual that has COVID-19 or is at risk for COVID-19 or is suspected of having COVID-19, or that is in need of preventing COVID-19.

### REFERENCES

1. Suresh, G., K. Haridasan, and K. V. Krishnamurthy, Relevance of Vrksayurveda and other traditional methods for organic production of nursery seedlings of useful plants. Anc Sci Life, 2013. 33(1): p. 60-70.
2. Oglesby, R. T., R. F. Christman, and C. H. Driver, The biotransformation of lignin to humus facts and postulates. Adv Appl Microbiol, 1967. 9: p. 171-84.
3. Huang, K. J., et al., An interferon-gamma-related cytokine storm in SARS patients. J Med Virol, 2005. 75(2): p. 185-94.
4. Theron, M., et al., A probable role for IFN-gamma in the development of a lung immunopathology in SARS. Cytokine, 2005. 32(1): p. 30-8.
5. Summerfield, A., et al., High IFN-alpha responses associated with depletion of lymphocytes and natural IFN-producing cells during classical swine fever. J Interferon Cytokine Res, 2006. 26(4): p. 248-55.
6. Clark, I. A., The advent of the cytokine storm. Immunol Cell Biol, 2007. 85(4): p. 271-3.
7. London, N. R., et al., Targeting Robo4-dependent Slit signaling to survive the cytokine storm in sepsis and influenza. Sci Transl Med, 2010. 2(23): p. 23ra19.
8. Cheng, X. W., et al., Three fatal cases of pandemic 2009 influenza A virus infection in Shenzhen are associated with cytokine storm. Respir Physiol Neurobiol, 2011. 175(1): p. 185-7.
9. Nakamura, R., et al., Interleukin-15 is critical in the pathogenesis of influenza a virus-induced acute lung injury. J Virol, 2010. 84(11): p. 5574-82.
10. Walsh, K. B., et al., Quelling the storm: utilization of sphingosine-1-phosphate receptor signaling to ameliorate influenza virus-induced cytokine storm. Immunol Res, 2011. 51(1): p. 15-25.
11. Teijaro, J. R., et al., Endothelial cells are central orchestrators of cytokine amplification during influenza virus infection. Cell, 2011. 146(6): p. 980-91.
12. Tisoncik, J. R., et al., Into the eye of the cytokine storm. Microbiol Mol Biol Rev, 2012. 76(1): p. 16-32.
13. D'Elia, R. V., et al., Targeting the “cytokine storm” for therapeutic benefit. Clin Vaccine Immunol, 2013. 20(3): p. 319-27.
14. Dutta, A., et al., Altered T-bet dominance in IFN-gamma-decoupled CD4+ T cells with attenuated cytokine storm and preserved memory in influenza. J Immunol, 2013. 190(8): p. 4205-14.
15. van de Weg, C. A., et al., Microbial translocation is associated with extensive immune activation in dengue virus infected patients with severe disease. PLoS Neg1 Trop Dis, 2013. 7(5): p. e2236.
16. Yang, Y. and H. Tang, Aberrant coagulation causes a hyper-inflammatory response in severe influenza pneumonia. Cell Mol Immunol, 2016. 13(4): p. 432-42.
17. Davison, A. M., D. Thomson, and J. S. Robson, Intravascular coagulation complicating influenza A virus infection. Br Med J, 1973. 1(5854): p. 654-5.
18. Wauquier, N., et al., Human fatal zaire ebola virus infection is associated with an aberrant innate immunity and with massive lymphocyte apoptosis. PLoS Neg1 Trop Dis, 2010. 4(10).
19. Muller, N., et al., A CD28 superagonistic antibody elicits 2 functionally distinct waves of T cell activation in rats. J Clin Invest, 2008. 118(4): p. 1405-16.
20. St Clair, E. W., The calm after the cytokine storm: lessons from the TGN1412 trial. J Clin Invest, 2008. 118(4): p. 1344-7.
21. Giamarellos-Bourboulis, E. J., What is the pathophysiology of the septic host upon admission? Int J Antimicrob Agents, 2010. 36 Suppl 2: p. S2-5.
22. Francois, B., et al., Interleukin-7 restores lymphocytes in septic shock: the IRIS-7 randomized clinical trial. JCI Insight, 2018. 3(5).
23. Hawkins, R. B., et al., Chronic Critical Illness and the Persistent Inflammation, Immunosuppression, and Catabolism Syndrome. Front Immunol, 2018. 9: p. 1511.
24. Carvelli, J., et al., Imbalance of Circulating Innate Lymphoid Cell Subpopulations in Patients With Septic Shock. Front Immunol, 2019. 10: p. 2179.
25. Chollet-Martin, S., et al., High levels of interleukin-8 in the blood and alveolar spaces of patients with pneumonia and adult respiratory distress syndrome. Infect Immun, 1993. 61(11): p. 4553-9.
26. Groeneveld, A. B., et al., Interleukin 8-related neutrophil elastase and the severity of the adult respiratory distress syndrome. Cytokine, 1995. 7(7): p. 746-52.
27. Villard, J., et al., GRO alpha and interleukin-8 in Pneumocystis carinii or bacterial pneumonia and adult respiratory distress syndrome. Am J Respir Crit Care Med, 1995. 152(5 Pt 1): p. 1549-54.
28. Miller, E. J., A. B. Cohen, and M. A. Matthay, Increased interleukin-8 concentrations in the pulmonary edema fluid of patients with acute respiratory distress syndrome from sepsis. Crit Care Med, 1996. 24(9): p. 1448-54.
29. Yokoi, K., et al., Prevention of endotoxemia-induced acute respiratory distress syndrome-like lung injury in rabbits by a monoclonal antibody to IL-8. Lab Invest, 1997. 76(3): p. 375-84.
30. Goodman, E. R., et al., Role of interleukin 8 in the genesis of acute respiratory distress syndrome through an effect on neutrophil apoptosis. Arch Surg, 1998. 133(11): p. 1234-9.
31. Mukaida, N., et al., Inhibition of neutrophil-mediated acute inflammation injury by an antibody against interleukin-8 (IL-8). Inflamm Res, 1998. 47 Suppl 3: p. S151-7.
32. Pugin, J., et al., The alveolar space is the site of intense inflammatory and profibrotic reactions in the early phase of acute respiratory distress syndrome. Crit Care Med, 1999. 27(2): p. 304-12.
33. Amat, M., et al., Evolution of leukotriene B4, peptide leukotrienes, and interleukin-8 plasma concentrations in patients at risk of acute respiratory distress syndrome and with acute respiratory distress syndrome: mortality prognostic study. Crit Care Med, 2000. 28(1): p. 57-62.
34. Aggarwal, A., et al., G-CSF and IL-8 but not GM-CSF correlate with severity of pulmonary neutrophilia in acute respiratory distress syndrome. Eur Respir J, 2000. 15(5): p. 895-901.
35. Dunican, A. L., et al., TNFalpha-induced suppression of PMN apoptosis is mediated through interleukin-8 production. Shock, 2000. 14(3): p. 284-8; discussion 288-9.
36. Abul, H., et al., Levels of IL-8 and myeloperoxidase in the lungs of pneumonia patients. Mol Cell Biochem, 2001. 217(1-2): p. 107-12.
37. Hirani, N., et al., The regulation of interleukin-8 by hypoxia in human macrophages—a potential role in the pathogenesis of the acute respiratory distress syndrome (ARDS). Mol Med, 2001. 7(10): p. 685-97.
38. Hildebrand, F., et al., Association of IL-8-251A/T polymorphism with incidence of Acute Respiratory Distress Syndrome (ARDS) and IL-8 synthesis after multiple trauma. Cytokine, 2007. 37(3): p. 192-9.
39. Fudala, R., et al., Anti-IL-8 autoantibody: IL-8 immune complexes suppress spontaneous apoptosis of neutrophils. Am J Physiol Lung Cell Mol Physiol, 2007. 293(2): p. L364-74.
40. Chollet-Martin, S., et al., Interactions between neutrophils and cytokines in blood and alveolar spaces during ARDS. Am J Respir Crit Care Med, 1996. 154(3 Pt 1): p. 594-601.
41. Meduri, G. U., et al., Persistent elevation of inflammatory cytokines predicts a poor outcome in ARDS. Plasma IL-1 beta and IL-6 levels are consistent and efficient predictors of outcome over time. Chest, 1995. 107(4): p. 1062-73.
42. Goodman, R. B., et al., Inflammatory cytokines in patients with persistence of the acute respiratory distress syndrome. Am J Respir Crit Care Med, 1996. 154(3 Pt 1): p. 602-11.
43. Chada, M., et al., Anakinra (IL-1R antagonist) lowers pulmonary artery pressure in a neonatal surfactant depleted piglet model. Pediatr Pulmonol, 2008. 43(9): p. 851-7.
44. Kobayashi, A., et al., Expression of inducible nitric oxide synthase and inflammatory cytokines in alveolar macrophages of ARDS following sepsis. Chest, 1998. 113(6): p. 1632-9.
45. Tagami, T., et al., Effect of a selective neutrophil elastase inhibitor on mortality and ventilator-free days in patients with increased extravascular lung water: a post hoc analysis of the PiCCO Pulmonary Edema Study. J Intensive Care, 2014. 2(1): p. 67.
46. Engel, W. K., Intravenous immunoglobulin G is remarkably beneficial in chronic immune dysschwannian/dysneuronal polyneuropathy, diabetes-2 neuropathy, and potentially in severe acute respiratory syndrome. Acta Myol, 2003. 22(3): p. 97-103.
47. Hagiwara, S., et al., High-dose intravenous immunoglobulin G improves systemic inflammation in a rat model of CLP-induced sepsis. Intensive Care Med, 2008. 34(10): p. 1812-9.
48. Chong, J. L., S. Sapari, and Y. C. Kuan, A case of acute respiratory distress syndrome associated with novel H1N1 treated with intravenous immunoglobulin G. J Microbiol Immunol Infect, 2011. 44(4): p. 319-22.
49. Bao, Z., et al., Humanized monoclonal antibody against the chemokine CXCL-8 (IL-8) effectively prevents acute lung injury. Int Immunopharmacol, 2010. 10(2): p. 259-63.
50. Vincent, J. L., et al., A multi-centre, double-blind, placebo-controlled study of liposomal prostaglandin E1 (TLC C-53) in patients with acute respiratory distress syndrome. Intensive Care Med, 2001. 27(10): p. 1578-83.
51. Saitoh, H., et al., Effect of antisense oligonucleotides to nuclear factor-kappaB on the survival of LPS-induced ARDS in mouse. Exp Lung Res, 2002. 28(3): p. 219-31.
52. Weiss, Y. G., et al., Adenoviral transfer of HSP-70 into pulmonary epithelium ameliorates experimental acute respiratory distress syndrome. J Clin Invest, 2002. 110(6): p. 801-6.
53. Schuster, D. P., et al., Recombinant platelet-activating factor acetylhydrolase to prevent acute respiratory distress syndrome and mortality in severe sepsis: Phase IIb, multicenter, randomized, placebo-controlled, clinical trial. Crit Care Med, 2003. 31(6): p. 1612-9.
54. Ohbayashi, H., Novel neutrophil elastase inhibitors as a treatment for neutrophil-predominant inflammatory lung diseases. IDrugs, 2002. 5(9): p. 910-23.
55. Kadoi, Y., et al., Pilot study of the effects of ONO-5046 in patients with acute respiratory distress syndrome. Anesth Analg, 2004. 99(3): p. 872-7, table of contents.
56. Spragg, R. G., et al., Effect of recombinant surfactant protein C-based surfactant on the acute respiratory distress syndrome. N Engl J Med, 2004. 351(9): p. 884-92.
57. Singleton, K. D., V. E. Beckey, and P. E. Wischmeyer, GLUTAMINE PREVENTS ACTIVATION OF NF-kappaB AND STRESS KINASE PATHWAYS, ATTENUATES INFLAMMATORY CYTOKINE RELEASE, AND PREVENTS ACUTE RESPIRATORY DISTRESS SYNDROME (ARDS) FOLLOWING SEPSIS. Shock, 2005. 24(6): p. 583-9.
58. Lai, C. C., W. L. Liu, and C. M. Chen, Glutamine attenuates acute lung injury caused by acid aspiration. Nutrients, 2014. 6(8): p. 3101-16.
59. Chen, C. M., et al., The protective effects of glutamine in a rat model of ventilator-induced lung injury. J Thorac Dis, 2014. 6(12): p. 1704-13.
60. Ryffel, B., et al., Histamine scavenging attenuates endotoxin-induced acute lung injury. Ann N Y Acad Sci, 2005. 1056: p. 197-205.
61. Yeh, C. C., et al., The immunomodulation of endotoxin-induced acute lung injury by hesperidin in vivo and in vitro. Life Sci, 2007. 80(20): p. 1821-31.
62. Bedirli, A., et al., Beta-glucan attenuates inflammatory cytokine release and prevents acute lung injury in an experimental model of sepsis. Shock, 2007. 27(4): p. 397-401.
63. Heslet, L., et al., Inhalation of activated protein C: A possible new adjunctive intervention in acute respiratory distress syndrome. Biologics, 2007. 1(4): p. 465-72.
64. Cornet, A. D., et al., Activated protein C in the treatment of acute lung injury and acute respiratory distress syndrome. Expert Opin Drug Discov, 2009. 4(3): p. 219-27.
65. Kallet, R. H., R. M. Jasmer, and J. F. Pittet, Alveolar dead-space response to activated protein C in acute respiratory distress syndrome. Respir Care, 2010. 55(5): p. 617-22.
66. Xie, Y. C., et al., Inhibitory effects of flavonoids extracted from licorice on lipopolysaccharide-induced acute pulmonary inflammation in mice. Int Immunopharmacol, 2009. 9(2): p. 194-200.
67. Liu, D., et al., Suppression of acute lung inflammation by intracellular peptide delivery of a nuclear import inhibitor. Mol Ther, 2009. 17(5): p. 796-802.
68. Kono, K., et al., Direct hemoperfusion with a beta2-microglobulin-selective adsorbent column eliminates inflammatory cytokines and improves pulmonary oxygenation. Ther Apher Dial, 2009. 13(1): p. 27-33.
69. Nakamura, T., et al., Effect of polymyxin B-immobilized fiber hemoperfusion on serum high mobility group box-1 protein levels and oxidative stress in patients with acute respiratory distress syndrome. ASAIO J, 2009. 55(4): p. 395-9.
70. Li, G., et al., Growth hormone releasing peptide-2, a ghrelin agonist, attenuates lipopolysaccharide-induced acute lung injury in rats. Tohoku J Exp Med, 2010. 222(1): p. 7-13.
71. Kakavas, S., et al., Erythropoetin as a novel agent with pleiotropic effects against acute lung injury. Eur J Clin Pharmacol, 2011. 67(1): p. 1-9.
72. Li, H., et al., Effects of early administration of a novel anticholinergic drug on acute respiratory distress syndrome induced by sepsis. Med Sci Monit, 2011. 17(11): p. BR319-325.
73. Fang, X., C. Bai, and X. Wang, Potential clinical application of KGF-2 (FGF-10) for acute lung injury/acute respiratory distress syndrome. Expert Rev Clin Pharmacol, 2010. 3(6): p. 797-805.
74. Morris, P. E., et al., A phase I study evaluating the pharmacokinetics, safety and tolerability of an antibody-based tissue factor antagonist in subjects with acute lung injury or acute respiratory distress syndrome. BMC Pulm Med, 2012. 12: p. 5.
75. Fu, P. K., et al., Anti-inflammatory and anticoagulative effects of paeonol on LPS-induced acute lung injury in rats. Evid Based Complement Alternat Med, 2012. 2012: p. 837513.
76. Hartmann, E. K., et al., An inhaled tumor necrosis factor-alpha-derived TIP peptide improves the pulmonary function in experimental lung injury. Acta Anaesthesiol Scand, 2013. 57(3): p. 334-41.
77. Shi, Y., et al., Osthole protects lipopolysaccharide-induced acute lung injury in mice by preventing down-regulation of angiotensin-converting enzyme 2. Eur J Pharm Sci, 2013. 48(4-5): p. 819-24.
78. Kudo, D., et al., Low-dose interferon-alpha treatment improves survival and inflammatory responses in a mouse model of fulminant acute respiratory distress syndrome. Inflammation, 2013. 36(4): p. 812-20.
79. Avasarala, S., et al., Curcumin modulates the inflammatory response and inhibits subsequent fibrosis in a mouse model of viral-induced acute respiratory distress syndrome. PLoS One, 2013. 8(2): p. e57285.
80. Sharifov, O. F., et al., Anti-inflammatory mechanisms of apolipoprotein A-I mimetic peptide in acute respiratory distress syndrome secondary to sepsis. PLoS One, 2013. 8(5): p. e64486.
81. Mehla, K., et al., Ethyl gallate attenuates acute lung injury through Nrf2 signaling. Biochimie, 2013. 95(12): p. 2404-14.
82. Britt, R. D., Jr., et al., The thioredoxin reductase-1 inhibitor aurothioglucose attenuates lung injury and improves survival in a murine model of acute respiratory distress syndrome. Antioxid Redox Signal, 2014. 20(17): p. 2681-91.
83. Gonzales, J. N., et al., Protective effect of adenosine receptors against lipopolysaccharide-induced acute lung injury. Am J Physiol Lung Cell Mol Physiol, 2014. 306(6): p. L497-507.
84. Zhou, E., et al., Schisantherin A protects lipopolysaccharide-induced acute respiratory distress syndrome in mice through inhibiting NF-kappaB and MAPKs signaling pathways. Int Immunopharmacol, 2014. 22(1): p. 133-40.
85. Peng, J., et al., Punicalagin ameliorates lipopolysaccharide-induced acute respiratory distress syndrome in mice. Inflammation, 2015. 38(2): p. 493-9.
86. Xiao, M., et al., Emodin ameliorates LPS-induced acute lung injury, involving the inactivation of NF-kappaB in mice. Int J Mol Sci, 2014. 15(11): p. 19355-68.
87. Zhu, T., et al., Emodin suppresses LPS-induced inflammation in RAW264.7 cells through a PPARgamma-dependent pathway. Int Immunopharmacol, 2016. 34: p. 16-24.
88. Takashima, K., et al., Protective effects of intratracheally administered quercetin on lipopolysaccharide-induced acute lung injury. Respir Res, 2014. 15: p. 150.
89. Stephens, R. S., et al., The tyrosine kinase inhibitor imatinib prevents lung injury and death after intravenous LPS in mice. Physiol Rep, 2015. 3(11).
90. Zhang, Y., et al., Melatonin alleviates acute lung injury through inhibiting the NLRP3 inflammasome. J Pineal Res, 2016. 60(4): p. 405-14.
91. Guo, C., et al., A Stromal Cell-Derived Factor 1alpha Analogue Improves Endothelial Cell Function in Lipopolysaccharide-Induced Acute Respiratory Distress Syndrome. Mol Med, 2016. 22: p. 115-123.
92. Kong, G., et al., Astilbin alleviates LPS-induced ARDS by suppressing MAPK signaling pathway and protecting pulmonary endothelial glycocalyx. Int Immunopharmacol, 2016. 36: p. 51-58.
93. Li, G. G., et al., Inhibition of CD8(+) T cells and elimination of myeloid cells by CD4(+) Foxp3(−) T regulatory type 1 cells in acute respiratory distress syndrome. Clin Exp Pharmacol Physiol, 2016. 43(12): p. 1191-1198.
94. Zhao, J., et al., Protective effect of suppressing STAT3 activity in LPS-induced acute lung injury. Am J Physiol Lung Cell Mol Physiol, 2016. 311(5): p. L868-L880.
95. Bharara, A., et al., Intravenous Vitamin C Administered as Adjunctive Therapy for Recurrent Acute Respiratory Distress Syndrome. Case Rep Crit Care, 2016. 2016: p. 8560871.
96. Lang, S., et al., CXCL10/IP-10 Neutralization Can Ameliorate Lipopolysaccharide-Induced Acute Respiratory Distress Syndrome in Rats. PLoS One, 2017. 12(1): p. e0169100.
97. Li, C., et al., Lugrandoside attenuates LPS-induced acute respiratory distress syndrome by anti-inflammation and anti-apoptosis in mice. Am J Transl Res, 2016. 8(12): p. 5557-5568.
98. Zhang, Y., et al., Hydroxysafflor Yellow A Alleviates Lipopolysaccharide-Induced Acute Respiratory Distress Syndrome in Mice. Biol Pharm Bull, 2017. 40(2): p. 135-144.
99. Meng, F. Y., W. Gao, and Y. N. Ju, Parecoxib reduced ventilation induced lung injury in acute respiratory distress syndrome. BMC Pharmacol Toxicol, 2017. 18(1): p. 25.
100. Liu, Q., et al., Diosmetin Alleviates Lipopolysaccharide-Induced Acute Lung Injury through Activating the Nrf2 Pathway and Inhibiting the NLRP3 Inflammasome. Biomol Ther (Seoul), 2018. 26(2): p. 157-166.
101. Svedova, J., et al., Therapeutic blockade of CD54 attenuates pulmonary barrier damage in T cell-induced acute lung injury. Am J Physiol Lung Cell Mol Physiol, 2017. 313(1): p. L177-L191.
102. Malaviya, R., J. D. Laskin, and D. L. Laskin, Anti-TNFalpha therapy in inflammatory lung diseases. Pharmacol Ther, 2017. 180: p. 90-98.
103. Wang, C., et al., Geldanamycin Reduces Acute Respiratory Distress Syndrome and Promotes the Survival of Mice Infected with the Highly Virulent H5N1 Influenza Virus. Front Cell Infect Microbiol, 2017. 7: p. 267.
104. He, J., et al., Effect of rhubarb on extravascular lung water in patients with acute respiratory distress syndrome. Rev Assoc Med Bras (1992), 2017. 63(5): p. 435-440.
105. Wu, D. Q., et al., Effects of Zinc Finger Protein A20 on Lipopolysaccharide (LPS)-Induced Pulmonary Inflammation/Anti-Inflammatory Mediators in an Acute Lung Injury/Acute Respiratory Distress Syndrome Rat Model. Med Sci Monit, 2017. 23: p. 3536-3545.
106. Gao, Y., et al., Resolvin D1 Improves the Resolution of Inflammation via Activating NF-kappaB p50/p50-Mediated Cyclooxygenase-2 Expression in Acute Respiratory Distress Syndrome. J Immunol, 2017.
107. Ding, Q., et al., Glycogen synthase kinase3beta inhibitor reduces LPS-induced acute lung injury in mice. Mol Med Rep, 2017. 16(5): p. 6715-6721.
108. Qi, D., et al., Vaspin protects against LPS-induced ARDS by inhibiting inflammation, apoptosis and reactive oxygen species generation in pulmonary endothelial cells via the Akt/GSK3beta pathway. Int J Mol Med, 2017. 40(6): p. 1803-1817.
109. Kim, K. D., et al., Adaptive immune cells temper initial innate responses. Nat Med, 2007. 13(10): p. 1248-52.
110. Yokoyama, T., et al., A case of severe ARDS caused by novel swine-origin influenza (A/H1N1pdm) virus: a successful treatment with direct hemoperfusion with polymyxin B-immobilized fiber. J Clin Apher, 2010. 25(6): p. 350-3.
111. Li, R., et al., Attenuated Bordetella pertussis protects against highly pathogenic influenza A viruses by dampening the cytokine storm. J Virol, 2010. 84(14): p. 7105-13.
112. Matheu, M. P., et al., Three phases of CD8 T cell response in the lung following H1N1 influenza infection and sphingosine 1 phosphate agonist therapy. PLoS One, 2013. 8(3): p. e58033.
113. Lomas, D. A., et al., Inhibition of plasmin, urokinase, tissue plasminogen activator, and C1S by a myxoma virus serine proteinase inhibitor. J Biol Chem, 1993. 268(1): p. 516-21.
114. McFadden, G., et al., Interruption of cytokine networks by poxviruses: lessons from myxoma virus. J Leukoc Biol, 1995. 57(5): p. 731-8.
115. Maksymowych, W. P., et al., Amelioration of antigen induced arthritis in rabbits treated with a secreted viral serine proteinase inhibitor. J Rheumatol, 1996. 23(5): p. 878-82.
116. Nash, P., A. Lucas, and G. McFadden, SERP-1, a poxvirus-encoded serpin, is expressed as a secreted glycoprotein that inhibits the inflammatory response to myxoma virus infection. Adv Exp Med Biol, 1997. 425: p. 195-205.
117. Dai, E., et al., Identification of myxomaviral serpin reactive site loop sequences that regulate innate immune responses. J Biol Chem, 2006. 281(12): p. 8041-50.
118. Bedard, E. L., et al., Prevention of chronic renal allograft rejection by SERP-1 protein. Transplantation, 2006. 81(6): p. 908-14.
119. Viswanathan, K., et al., Myxoma viral serpin, Serp-1, inhibits human monocyte adhesion through regulation of actin-binding protein filamin B. J Leukoc Biol, 2009. 85(3): p. 418-26.
120. Tardif, J. C., et al., A randomized controlled, phase 2 trial of the viral serpin Serp-1 in patients with acute coronary syndromes undergoing percutaneous coronary intervention. Circ Cardiovasc Interv, 2010. 3(6): p. 543-8.
121. Spiesschaert, B., et al., The current status and future directions of myxoma virus, a master in immune evasion. Vet Res, 2011. 42: p. 76.
122. Chen, H., et al., Viral serpin therapeutics from concept to clinic. Methods Enzymol, 2011. 499: p. 301-29.
123. Brahn, E., et al., Suppression of collagen-induced arthritis with a serine proteinase inhibitor (serpin) derived from myxoma virus. Clin Immunol, 2014. 153(2): p. 254-63.
124. Lucas, A. R., et al., Myxomavirus anti-inflammatory chemokine binding protein reduces the increased plaque growth induced by chronic Porphyromonas gingivalis oral infection after balloon angioplasty aortic injury in mice. PLoS One, 2014. 9(10): p. e111353.
125. Kwiecien, J. M., et al., Myxoma virus derived immune modulating proteins, M-T7 and Serp-1, reduce early inflammation after spinal cord injury in the rat model. Folia Neuropathol, 2019. 57(1): p. 41-50.
126. Nash, P., et al., Inhibitory specificity of the anti-inflammatory myxoma virus serpin, SERP-1. J Biol Chem, 1998. 273(33): p. 20982-91.
127. Miller, L. W., et al., Inhibition of transplant vasculopathy in a rat aortic allograft model after infusion of anti-inflammatory viral serpin. Circulation, 2000. 101(13): p. 1598-605.
128. Lucas, A., et al., Transplant vasculopathy: viral anti-inflammatory serpin regulation of atherogenesis. J Heart Lung Transplant, 2000. 19(11): p. 1029-38.
129. Bot, I., et al., Serine protease inhibitor Serp-1 strongly impairs atherosclerotic lesion formation and induces a stable plaque phenotype in ApoE−/−mice. Circ Res, 2003. 93(5): p. 464-71.
130. Richardson, J., K. Viswanathan, and A. Lucas, Serpins, the vasculature, and viral therapeutics. Front Biosci, 2006. 11: p. 1042-56.
131. Hatton, M. W., et al., Metabolism and distribution of the virus-encoded serine proteinase inhibitor SERP-1 in healthy rabbits. Metabolism, 2000. 49(11): p. 1449-52.
132. Li, X., et al., Heparin alters viral serpin, serp-1, anti-thrombolytic activity to anti-thrombotic activity. Open Biochem J, 2008. 2: p. 6-15.
133. Mahon, B. P., et al., Crystal Structure of Cleaved Serp-1, a Myxomavirus-Derived Immune Modulating Serpin: Structural Design of Serpin Reactive Center Loop Peptides with Improved Therapeutic Function. Biochemistry, 2018. 57(7): p. 1096-1107.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of treating excessive production of one or more cytokines in an individual, comprising the step of administering to the individual a therapeutically effective amount of Sphagnum, Sphagnum extract, a preparation of Sphagnum, a composition derived from Sphagnum, or a combination thereof.

2. The method of claim 1, wherein the Sphagnum extract is an ethanol extract of Sphagnum, a methanol extract of Sphagnum and/or an aqueous extract of Sphagnum.

3. The method of claim 1 or claim 2, wherein the preparation of Sphagnum comprises tolpa peat preparation.

4. The method of any one of claims 1-3, wherein the composition derived from Sphagnum comprises humic acid and/or fulvic acid.

5. The method of any one of claims 1-4, wherein an additional therapy is administered to the individual, wherein the additional therapy comprises an antiviral therapy, an immunosuppressive therapy, a chelating agent, an NF-kappa B inhibitor, an antimalarial therapy, a cellular therapy, or a combination thereof.

6. The method of claim 5, wherein the antiviral therapy comprises hydroxychloroquine and/or chloroquine.

7. The method of claim 5, wherein the immunosuppressive therapy comprises rapamycin, cyclophosphamide, prednisone (Deltasone, Orasone), budesonide (Entocort EC), prednisolone (Millipred), tofacitinib (Xeljanz), cyclosporine (Neoral, Sandimmune, SangCya), tacrolimus (Astagraf XL, Envarsus XR, Prograf), mTOR inhibitors, sirolimus (Rapamune), everolimus (Afinitor, Zortress), IMDH inhibitors, azathioprine (Azasan, Imuran), leflunomide (Arava), mycophenolate (CellCept, Myfortic), abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), monoclonal antibodies, basiliximab (Simulect), daclizumab (Zinbryta), or a combination thereof.

8. The method of claim 5, wherein the chelating agent comprises deferoxamine mesylate.

9. The method of claim 5, wherein the NF-kappa B inhibitor comprises one or more antisense oligonucleotides, decoy oligonucleotides, short-hairpin RNAs, and/or RNA interference compositions targeting at least one gene in the NF-kappa B pathway.

10. The method of claim 5, wherein the NF-kappa B inhibitor is a composition selected from the group consisting of Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic), and a combination thereof.

11. The method of claim 5, wherein the cellular therapy comprises mesenchymal stem cells, hematopoietic stem cells, natural killer cells, and/or fibroblasts.

12. The method of claim 11, wherein the cellular therapy comprises cells that are autologous, allogenic, or xenogenic to the individual.

13. The method of claim 11, wherein the mesenchymal stem cells and/or fibroblasts are plastic adherent.

14. The method of claim 11, wherein the mesenchymal stem cells express one or more of CD73, CD90, and/or CD105.

15. The method of claim 11, wherein the mesenchymal stem cells do not express one or more of CD14, CD34, and/or HLA II.

16. The method of claim 11, wherein the hematopoietic stem cells express CD34 and/or CD133.

17. The method of claim 11, wherein the hematopoietic stem cells do not express CD38.

18. The method of claim 11, wherein the hematopoietic stem cells are capable of differentiating into myeloid, erythroid, and/or megakaryocytic lineages.

19. The method of claim 11, wherein the fibroblasts are derived from tissue selected from the group consisting of skin, fat, bone marrow, cord blood, Wharton's jelly, hair follicle, and a combination thereof.

20. The method of any one of claims 1-19, wherein the individual is additionally administered a composition selected from the group consisting of remdesivir, pyridostigmine, desferal, hyperimmune plasma, toclizumab, sialidase DAS181, Dapagliflozin, recombinant ACE2, naproxen, Lopinavir/ritonavir, Baricitinib (Janus kinase inhibitor), Sarilumab (anti-IL-6 receptor), Ruxolitinib, Acalabrutinib, interferon, Ciclesonide, Anakinra, Umifenovir, Sargramostim, Sildenafil citrate, Tranexamic acid, Ivermectine, myxoma virus SERPIN-1 protein, and a combination thereof.

21. The method of any one of claims 1-20, wherein the excessive production of cytokines is mediated by unrestrained activation of cells selected from the group consisting of monocytes, peripheral blood mononuclear cells, dendritic cells, gamma delta T cells, natural killer cells, and a combination thereof.

22. The method of any one of claims 1-21, wherein the excessive production of cytokines is not significantly controlled by anti-inflammatory cytokines.

23. The method of any one of claims 1-22, wherein the excessive production of cytokines comprises the production above physiological levels of cytokines selected from the group consisting of MCP-1, interleukin 1 beta, interleukin 6, interleukin 8, interleukin 11, interleukin-18, interleukin-21, interleukin 27, interleukin 33, HMGB-1, TNF-alpha, and a combination thereof.

24. The method of any one of claims 1-23, wherein the excessive production of cytokines comprises cytokine storm.

25. The method of any one of claims 1-24, wherein the excessive production of cytokines occurs as a result of a viral infection.

26. The method of claim 25, wherein the viral infection is a coronavirus infection.

27. The method of claim 26, wherein the viral infection is an infection of SARS-CoV-2.

28. A method of preventing lung injury in an individual, comprising administering to the individual a therapeutically effective amount of Sphagnum, Sphagnum extract, a preparation of Sphagnum, a composition derived from Sphagnum, or a combination thereof.

29. The method of claim 28, wherein the Sphagnum extract is an ethanol extract of Sphagnum, a methanol extract of Sphagnum and/or an aqueous extract of Sphagnum.

30. The method of claim 28 or claim 29, wherein the preparation of Sphagnum comprises tolpa peat preparation.

31. The method of any one of claims 28-30, wherein the composition derived from Sphagnum comprises humic acid and/or fulvic acid.

32. The method of any one of claims 28-31, wherein an additional therapy is administered to the individual, wherein the additional therapy comprises an antiviral therapy, an immunosuppressive therapy, a chelating agent, an NF-kappa B inhibitor, an antimalarial therapy, a cellular therapy, or a combination thereof.

33. The method of claim 32, wherein the antiviral therapy comprises hydroxychloroquine and/or chloroquine.

34. The method of claim 32, wherein the immunosuppressive therapy comprises rapamycin, cyclophosphamide, prednisone (Deltasone, Orasone), budesonide (Entocort EC), prednisolone (Millipred), tofacitinib (Xeljanz), cyclosporine (Neoral, Sandimmune, SangCya), tacrolimus (Astagraf XL, Envarsus XR, Prograf), mTOR inhibitors, sirolimus (Rapamune), everolimus (Afinitor, Zortress), IMDH inhibitors, azathioprine (Azasan, Imuran), leflunomide (Arava), mycophenolate (CellCept, Myfortic), abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), monoclonal antibodies, basiliximab (Simulect), daclizumab (Zinbryta), or a combination thereof.

35. The method of claim 32, wherein the chelating agent comprises deferoxamine mesylate.

36. The method of claim 32, wherein the NF-kappa B inhibitor comprises one or more antisense oligonucleotides, decoy oligonucleotides, short-hairpin RNAs, and/or RNA interference compositions targeting at least one gene in the NF-kappa B pathway.

37. The method of claim 32, wherein the NF-kappa B inhibitor is a composition selected from the group consisting of Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic), and a combination thereof.

38. The method of claim 32, wherein the cellular therapy comprises mesenchymal stem cells, hematopoietic stem cells, natural killer cells, and/or fibroblasts.

39. The method of claim 38, wherein the cellular therapy comprises cells that are autologous, allogenic, or xenogenic to the individual.

40. The method of claim 38, wherein the mesenchymal stem cells and/or fibroblasts are plastic adherent.

41. The method of claim 38, wherein the mesenchymal stem cells express one or more of CD73, CD90, and/or CD105.

42. The method of claim 38, wherein the mesenchymal stem cells do not express one or more of CD14, CD34, and/or HLA II.

43. The method of claim 38, wherein the hematopoietic stem cells express CD34 and/or CD133.

44. The method of claim 38, wherein the hematopoietic stem cells do not express CD38.

45. The method of claim 38, wherein the hematopoietic stem cells are capable of differentiating into myeloid, erythroid, and/or megakaryocytic lineages.

46. The method of claim 38, wherein the fibroblasts are derived from tissue selected from the group consisting of skin, fat, bone marrow, cord blood, Wharton's jelly, hair follicle, and a combination thereof.

47. The method of anyone of claims 28-46, wherein the individual is additionally administered a composition selected from the group consisting of remdesivir, pyridostigmine, desferal, hyperimmune plasma, toclizumab, sialidase DAS181, Dapagliflozin, recombinant ACE2, naproxen, Lopinavir/ritonavir, Baricitinib (Janus kinase inhibitor), Sarilumab (anti-IL-6 receptor), Ruxolitinib, Acalabrutinib, interferon, Ciclesonide, Anakinra, Umifenovir, Sargramostim, Sildenafil citrate, Tranexamic acid, Ivermectine, myxoma virus SERPIN-1 protein, and a combination thereof.

48. A method of stimulating production of keratinocyte growth factor in an individual comprising administering to the individual a therapeutically effective amount of Sphagnum, Sphagnum extract, a preparation of Sphagnum, a composition derived from Sphagnum, or a combination thereof.

49. The method of claim 48, wherein the Sphagnum extract is an ethanol extract of Sphagnum, a methanol extract of Sphagnum and/or an aqueous extract of Sphagnum.

50. The method of claim 48 or claim 49, wherein the preparation of Sphagnum comprises tolpa peat preparation.

51. The method of any one of claims 48-50, wherein the composition derived from Sphagnum comprises humic acid and/or fulvic acid.

52. The method of any one of claims 48-51, wherein an additional therapy is administered to the individual, wherein the additional therapy comprises an antiviral therapy, an immunosuppressive therapy, a chelating agent, an NF-kappa B inhibitor, an antimalarial therapy, a cellular therapy, or a combination thereof.

53. The method of claim 52, wherein the antiviral therapy comprises hydroxychloroquine and/or chloroquine.

54. The method of claim 52, wherein the immunosuppressive therapy comprises rapamycin, cyclophosphamide, prednisone (Deltasone, Orasone), budesonide (Entocort EC), prednisolone (Millipred), tofacitinib (Xeljanz), cyclosporine (Neoral, Sandimmune, SangCya), tacrolimus (Astagraf XL, Envarsus XR, Prograf), mTOR inhibitors, sirolimus (Rapamune), everolimus (Afinitor, Zortress), IMDH inhibitors, azathioprine (Azasan, Imuran), leflunomide (Arava), mycophenolate (CellCept, Myfortic), abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), monoclonal antibodies, basiliximab (Simulect), daclizumab (Zinbryta), or a combination thereof.

55. The method of claim 52, wherein the chelating agent comprises deferoxamine mesylate.

56. The method of claim 52, wherein the NF-kappa B inhibitor comprises one or more antisense oligonucleotides, decoy oligonucleotides, short-hairpin RNAs, and/or RNA interference compositions targeting at least one gene in the NF-kappa B pathway.

57. The method of claim 52, wherein the NF-kappa B inhibitor is a composition selected from the group consisting of Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic), and a combination thereof.

58. The method of claim 52, wherein the cellular therapy comprises mesenchymal stem cells, hematopoietic stem cells, natural killer cells, and/or fibroblasts.

59. The method of claim 58, wherein the cellular therapy comprises cells that are autologous, allogenic, or xenogenic to the individual.

60. The method of claim 58, wherein the mesenchymal stem cells and/or fibroblasts are plastic adherent.

61. The method of claim 58, wherein the mesenchymal stem cells express one or more of CD73, CD90, and/or CD105.

62. The method of claim 58, wherein the mesenchymal stem cells do not express one or more of CD14, CD34, and/or HLA II.

63. The method of claim 58, wherein the hematopoietic stem cells express CD34 and/or CD133.

64. The method of claim 58, wherein the hematopoietic stem cells do not express CD38.

65. The method of claim 58, wherein the hematopoietic stem cells are capable of differentiating into myeloid, erythroid, and/or megakaryocytic lineages.

66. The method of claim 58, wherein the fibroblasts are derived from tissue selected from the group consisting of skin, fat, bone marrow, cord blood, Wharton's jelly, hair follicle, and a combination thereof.

67. The method of any one of claims 48-66, wherein the individual is additionally administered a composition selected from the group consisting of remdesivir, pyridostigmine, desferal, hyperimmune plasma, toclizumab, sialidase DAS181, Dapagliflozin, recombinant ACE2, naproxen, Lopinavir/ritonavir, Baricitinib (Janus kinase inhibitor), Sarilumab (anti-IL-6 receptor), Ruxolitinib, Acalabrutinib, interferon, Ciclesonide, Anakinra, Umifenovir, Sargramostim, Sildenafil citrate, Tranexamic acid, Ivermectine, myxoma virus SERPIN-1 protein, and a combination thereof.

68. A method of treating or preventing one or more coronaviruses in an individual, comprising administering a therapeutically effective amount of Sphagnum, Sphagnum extract, a preparation of Sphagnum, a composition derived from Sphagnum, or a combination thereof to the individual.

69. The method of claim 69, wherein the Sphagnum extract is an ethanol extract of Sphagnum, a methanol extract of Sphagnum and/or an aqueous extract of Sphagnum.

70. The method of claim 68 or claim 69, wherein the preparation of Sphagnum comprises tolpa peat preparation.

71. The method of any one of claims 68-70, wherein the composition derived from Sphagnum comprises humic acid and/or fulvic acid.

72. The method of any one of claims 68-71, wherein an additional therapy is administered to the individual, wherein the additional therapy comprises an antiviral therapy, an immunosuppressive therapy, a chelating agent, an NF-kappa B inhibitor, an antimalarial therapy, a cellular therapy, or a combination thereof.

73. The method of claim 72, wherein the antiviral therapy comprises hydroxychloroquine and/or chloroquine.

74. The method of claim 72, wherein the immunosuppressive therapy comprises rapamycin, cyclophosphamide, prednisone (Deltasone, Orasone), budesonide (Entocort EC), prednisolone (Millipred), tofacitinib (Xeljanz), cyclosporine (Neoral, Sandimmune, SangCya), tacrolimus (Astagraf XL, Envarsus XR, Prograf), mTOR inhibitors, sirolimus (Rapamune), everolimus (Afinitor, Zortress), IMDH inhibitors, azathioprine (Azasan, Imuran), leflunomide (Arava), mycophenolate (CellCept, Myfortic), abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), monoclonal antibodies, basiliximab (Simulect), daclizumab (Zinbryta), or a combination thereof.

75. The method of claim 72, wherein the chelating agent comprises deferoxamine mesylate.

76. The method of claim 72, wherein the NF-kappa B inhibitor comprises one or more antisense oligonucleotides, decoy oligonucleotides, short-hairpin RNAs, and/or RNA interference compositions targeting at least one gene in the NF-kappa B pathway.

77. The method of claim 72, wherein the NF-kappa B inhibitor is a composition selected from the group consisting of Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic), and a combination thereof.

78. The method of claim 72, wherein the cellular therapy comprises mesenchymal stem cells, hematopoietic stem cells, natural killer cells, and/or fibroblasts.

79. The method of claim 78, wherein the cellular therapy comprises cells that are autologous, allogenic, or xenogenic to the individual.

80. The method of claim 78, wherein the mesenchymal stem cells and/or fibroblasts are plastic adherent.

81. The method of claim 78, wherein the mesenchymal stem cells express one or more of CD73, CD90, and/or CD105.

82. The method of claim 78, wherein the mesenchymal stem cells do not express one or more of CD14, CD34, and/or HLA II.

83. The method of claim 78, wherein the hematopoietic stem cells express CD34 and/or CD133.

84. The method of claim 78, wherein the hematopoietic stem cells do not express CD38.

85. The method of claim 78, wherein the hematopoietic stem cells are capable of differentiating into myeloid, erythroid, and/or megakaryocytic lineages.

86. The method of claim 78, wherein the fibroblasts are derived from tissue selected from the group consisting of skin, fat, bone marrow, cord blood, Wharton's jelly, hair follicle, and a combination thereof.

87. The method of any one of claims 68-86, wherein the individual is additionally administered a composition selected from the group consisting of remdesivir, pyridostigmine, desferal, hyperimmune plasma, toclizumab, sialidase DAS181, Dapagliflozin, recombinant ACE2, naproxen, Lopinavir/ritonavir, Baricitinib (Janus kinase inhibitor), Sarilumab (anti-IL-6 receptor), Ruxolitinib, Acalabrutinib, interferon, Ciclesonide, Anakinra, Umifenovir, Sargramostim, Sildenafil citrate, Tranexamic acid, Ivermectine, myxoma virus SERPIN-1 protein, and a combination thereof.

88. The method of any one of claims 68-87, wherein the coronavirus comprises SARS-CoV-2

89. The method of claim 88, wherein the individual is diagnosed with COVID-19 and/or has tested positive for SARS-CoV-2.

90. The method of any one of claims 68-89, wherein the individual is asymptomatic.

91. The method of any one of claims 68-89, wherein the individual is symptomatic.

92. The method of claim 91, wherein the individual has one or more symptoms selected from the group consisting of fever, cough, chest pain, chest pressure, shortness of breath, difficulty breathing, chills, repeated shaking, muscle pain, headache, sore throat, new loss of taste, new loss of smell, new confusion, bluish lips and/or face, and a combination thereof.
